(19) United States
(12) Patent Application Publication
(10) Pub. No.: US 2016/0270904 A1
(43) Pub. Date: Sep. 22, 2016

(54) INSTRUMENT FOR UNROLLING CORNEAL TISSUE IN DESCEMET'S MEMBRANE ENDOTHELIAL KERATOPLASTY SURGERY

(71) Applicant: William B. Neusidl, Easton, PA (US)

(72) Inventor: William B. Neusidl, Easton, PA (US)

(21) Appl. No.: 15/076,635

(22) Filed: Mar. 21, 2016

Related U.S. Application Data
(60) Provisional application No. 62/135,707, filed on Mar. 20, 2015.

(51) Int. Cl. A61F 2/14

(52) U.S. Cl. CPC A61F 2/148

(57) ABSTRACT

A surgical instrument to be used in an corneal transplant surgical procedure known in the art as Descemet's Membrane Endothelial Keratoplasty (DMEK). Specifically, the instrument disclosed herein (NHL, Neusidl-Hannush Loop), is designed for the portion of the DMEK procedure where upon the transplantation of a very thin layer of endothelial cells and their basement membrane (DMEK graft), the transplanted material must be flattened. In this step of the DMEK procedure, the DMEK graft, may form a scroll or a configuration with two scrolled-up edges. The instrument may unfold and flatten the rolled up DMEK graft enabling the transplanted material to be in position to adhere to the posterior surface of the cornea. The instrument may enable the unfolding of the layer of Descemet's membrane and endothelial cells without injuring, damaging, or destroying these cells during the unfolding process as is sometimes the case with other surgical methods currently performed.

### CROSS-REFERENCE TO RELATED APPLICATIONS

This Non-Provisional Application claims the benefit of U.S. Provisional Application No. 62/135,707, filed Mar. 20, 2015, the entire contents of which is herein incorporated by reference.

### FIELD OF THE INVENTION

The invention relates to a surgical instrument used in a type of corneal tissue transplant surgery known as Descemet's Membrane Endothelial Keratoplasty.

### BACKGROUND ART

This disclosure generally relates to instruments used in ocular surgical procedures. Specifically, the present invention is a surgical instrument to be used in a corneal tissue transplant procedure known as Descemet's Membrane Endothelial Keratoplasty, classified in the group of procedures known in the art as Endothelial Keratoplasty (EK).

There are three main types of EK procedures commonly practiced in the art. One is known as Deep Lamellar Endothelial Keratoplasty (DLEK), in which the cornea's posterior portion is replaced by corneal tissue from a donor, this is a type of ocular surgery where the endothelial replacement is done through a limbal scleral incision leaving untouched the surface of the recipient's cornea. Another type of EK procedure is known in the art as Descemet's Stripping Endothelial Keratoplasty (DSEK) in which a portion of the Descemet's membrane is taken out to be swapped for healthy posterior corneal tissue from a donor. The third type of EK is known as Descemet's Membrane Endothelial Keratoplasty (DMEK), this procedure is the latest of the EK procedures described above. DMEK involves the transplantation of a very thin layer of endothelial cells and their basement membrane from a donor's eye to that of a recipient. This surgical procedure evolved from DSEK. The major difference between the DMEK and DSEK procedures is the thickness of the layer of transplanted cell. In DSEK, the layer is approximately 100-150 microns, whereas in the DMEK procedure the transplanted layer is approximately 20 microns thick.

In the DMEK procedure when the thin layer of endothelial cells and their basement membrane is removed from a donor's cornea and transplanted into the anterior chamber of the recipient's eye, it forms a scroll similar to a cigar or a configuration with two scrolled up edges. One of the main obstacles a practitioner in the art has to overcome in the DMEK procedure is how to flatten out the transplanted tissue or unroll it so that it can adhere to the posterior surface of the recipient's cornea, without causing damage to the delicate endothelial cells in the “unscrolling ” maneuvers. At present, a practitioner has limited options available to open the rolled-up layer of endothelial cells and their basement membrane. These two layers comprise and are referred to as the “DMEK Graft”. The options for unrolling the transplanted tissue presently being practiced in the art include tapping repeatedly on the outer cornea, injecting air or fluid inside the eye, or manipulating the tissue further with surgical instrumentation. While these methods work, they are time consuming and could possibly damage the delicate endothelial cells as the tissue is floated around in the anterior chamber of the eye.

After transplantation, the instrument described herein may unfold and flatten the rolled up transplanted material, namely the endothelial cells and their basement membrane, once the tissue is inside the anterior chamber of the eye. This instrument, by correctly orienting the transplanted tissue, may enable the transplanted DMEK graft to adhere to the posterior surface of the cornea. It may also stabilize the tissue and may prevent it from floating away from where it is intended to be, centered in the anterior chamber of the recipients eye. The instrument may unroll the endothelial cell layer while reducing, minimizing, or eliminating damaging or destroying the cells during the transplant, as the currently practiced methods in the art are known to do.

### SUMMARY OF THE INVENTION

Disclosed is an apparatus to be used for unrolling the rolled up endothelial cells and their basement membrane used in the DMEK ocular surgery procedure. In an embodiment, a surgical instrument may have:

In an embodiment, an instrument for unrolling and flattening transplanted corneal tissue includes a handle housing at least a first actuator configured to advance in a distal direction and retract in a proximal direction by operation of a user, a distal tip for penetrating a corneal incision into an anterior chamber of the eye, at least one wire that may be fixed to a point on the distal tip on a distal end and fixed to the at least a first actuator on another end, wherein the at least a first actuator is advanced, the at least one wire expands in a horizontal plane forming a opened loop, and wherein the at least a first actuator is retracted, the at least one wire retracts closing said loop.

In another embodiment, the at least first actuator may be slidingly disposed inside said handle, the at least one wire is fixed to the at least one actuator at a point within said handle and is fixed to a point on the distal tip on a distal end outside of the handle, and the distal tip has an opening passing through the distal tip in a direction perpendicular to a field of motion of the at least one actuator.

In another embodiment, the at least one wire is fixed to a point on the distal tip by passing through an opening passing through said distal tip, and the at least one wire is fixed to a point on the at least one actuator at two ends of the at least one wire.

In yet another embodiment, the at least one wire expands in a horizontal plane forming an opened loop in a direction perpendicular to a field of motion of the at least one actuator, and the at least one wire is made of polypropylene or may be made of a medical grade suture.

In another embodiment, the distal tip may be further configured to being disposed in the center of transplanted corneal tissue scroll, and the corneal tissue scroll is unrolled when the at least one wire expands in a horizontal plane forming a opened loop, and the corneal tissue scroll is positioned against a posterior surface of the cornea when the at least one wire expands in a horizontal plane forming a opened loop, and the at least one wire retracts closing said loop allowing the distal tip to be removed from the anterior chamber of the eye.

In an embodiment, a device for unrolling descemet's membrane with endothelial cells in a DMEK procedure, said device including an elongated handle, generally cylindrical in shape and having a longitudinal axis, a distal end having an opening, an opposite proximal end, and a controller; the handle having a chamber extending lengthwise coaxial with the longitudinal axis of the handle, the chamber extending through to the opening at the distal end; an actuator that may be slidable via the controller in the chamber extending lengthwise coaxial with the longitudinal axis of the handle; a distal tip extending from the distal end of the handle; a wire, wherein two ends of the wire are in slidable communication with the actuator within the chamber and a length of the wire extends through the opening of the distal end of the handle; and wherein the length of wire is fixed to a point on the distal tip, such that the length of wire expands in a horizontal plane forming a opened loop when the actuator advances distally and the open loop is closed and a portion of the length of wire retracts into the handle when the actuator moves towards the proximal end of the handle.

In another embodiment, the distal tip is configured for penetrating a corneal incision into an anterior chamber of the eye; the length of wire is threaded through an opening in said distal tip, and the at least one wire expands in a horizontal plane forming an opened loop in a direction perpendicular to the longitudinal axis of said handle.

The instrument in all of its embodiments is designed to accommodate each or all of these configurations.

### BRIEF DESCRIPTION OF THE DRAWINGS

The present invention will be described by way of exemplary embodiments, illustrated in the accompanying figures in which like references denote similar elements.

FIG. 1 illustrates a perspective view of an instrument, in accordance with an embodiment of the present invention.

FIG. 2 illustrates a top view of an instrument, in accordance with an embodiment of the present invention.

FIG. 3 illustrates a detailed side view of a distal tip without a loop in place of an instrument, in accordance with an embodiment of the present invention.

FIG. 4 illustrates an elevation side view of a handle of an instrument, in accordance with an embodiment of the present invention.

FIG. 5 illustrates an elevation side view of a handle of an instrument beside a distal tip supported in a blister pack, in accordance with an embodiment of the present invention.

FIG. 6 illustrates a side elevation view of an embodiment of the instrument in an open loop position in accordance with an embodiment of the present invention.

FIG. 7 illustrates a side elevation view of an embodiment of the instrument in a closed loop position in accordance with an embodiment of the present invention.

FIG. 8 illustrates a cross-sectional side view of an embodiment of the instrument taken generally across line 8 in FIG. 2, in accordance with an embodiment of the present invention.

FIG. 9 illustrates a cross-sectional side view of a distal tip inserted into the piston of a handle of the instrument taken generally across line 8 in FIG. 2, in accordance with an embodiment of the present invention.

FIG. 10 illustrates a cross-sectional top view of an embodiment of the instrument taken generally across line 10 in FIG. 1, in accordance with an embodiment of the present invention.

FIG. 11 illustrates a detailed cross-sectional top view of a distal end of an embodiment of the instrument taken generally across line 10 in FIG. 1, in accordance with an embodiment of the present invention.

FIG. 12 illustrates a perspective view of a distal end of a handle of the instrument with the locking mechanism in the closed position, in accordance with an embodiment of the present invention.

FIG. 13 illustrates a perspective view of a distal end of a handle of the instrument with the locking mechanism in the open position, in accordance with an embodiment of the present invention.

FIG. 14A illustrates a top view of a blister pack showing the distal end of the instrument held in place in phantom lines, in accordance with an embodiment of the present invention.

FIG. 14B illustrates a side elevation view of a blister pack used with the instrument, in accordance with an embodiment of the present invention.

FIGS. 15A-15F illustrate the instrument in use in a procedure, in accordance with an embodiment of the present invention.

FIG. 16 illustrates a detailed side view of a distal tip without a loop in place of an instrument, in accordance with another embodiment of the present invention.

FIG. 17 illustrates a top view of an instrument, in accordance with another embodiment of the present invention.

### DETAILED DESCRIPTION OF THE DRAWINGS

Various aspects of the illustrative embodiments will be described using terms commonly employed by those skilled in the art to convey the substance of their work to other skilled practitioners. However, it will be apparent to those skilled in the art that the invention may be practiced with only some of the described aspects. For the purposes of explanation, specific numbers, materials and configurations are set forth in order to provide a thorough understanding of the illustrative embodiments. However, it will be apparent to one skilled in the art that the present invention may be practiced without the specific illustrative details described in any of the embodiments discussed here. In other instances, well known features are omitted or simplified in order not to obscure the illustrative embodiments.

Various operations will be described as multiple discrete elements, in a manner that is most helpful in understanding the present invention. However, the order of description should not be construed as to imply that these operations and elements are necessarily order dependent. In particular, these operations need not be performed in the order presented in any of the described embodiments.

The phrase “in an embodiment” is used repeatedly. The phrase does not necessarily refer to the same embodiment. The terms “comprising”, “having” and “including” are synonymous, unless the context dictates otherwise.

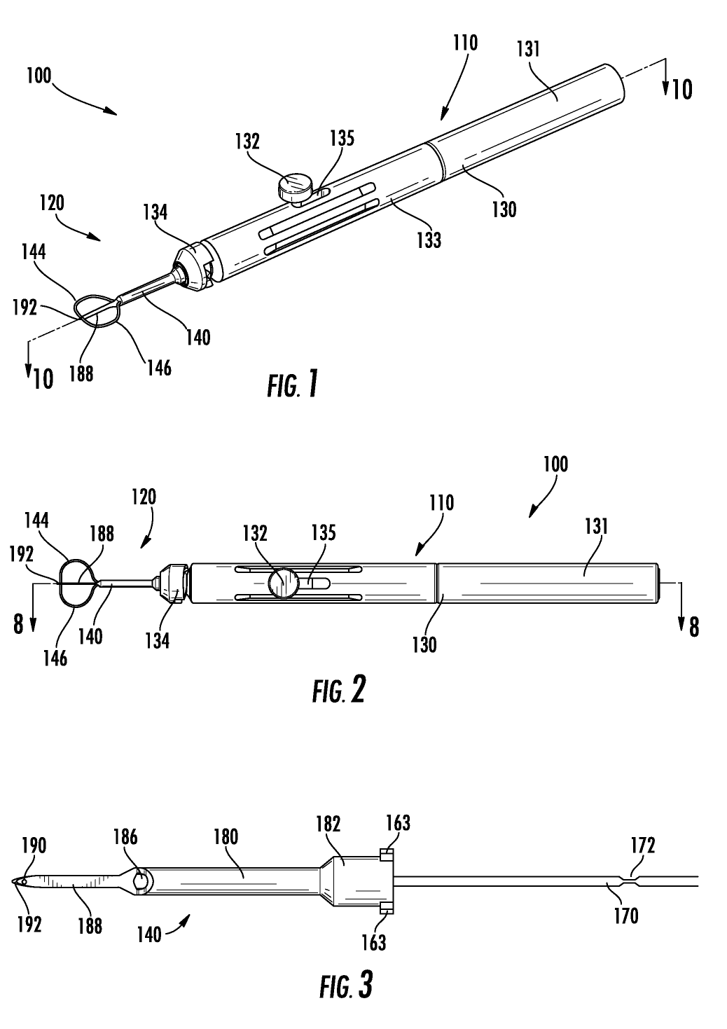

FIG. 1 shows an illustrative embodiment of the invention is an instrument 100 which may be used in DMEK surgery to unroll the scrolled DMEK graft, the layer of Descemet's membrane with its attached layer of endothelial cells is inserted into the anterior chamber of the eye.

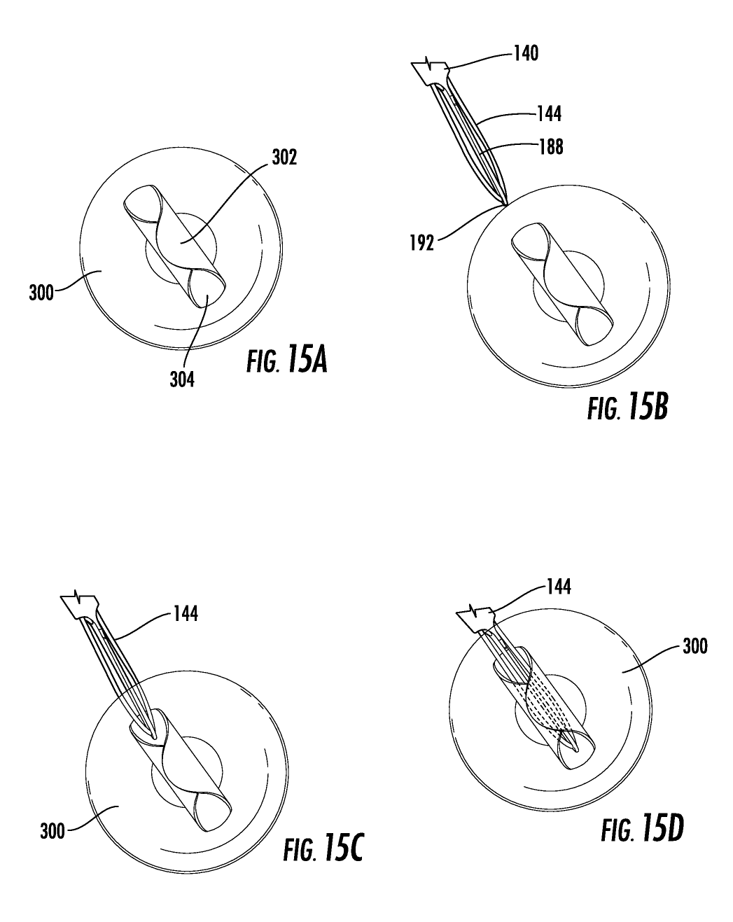

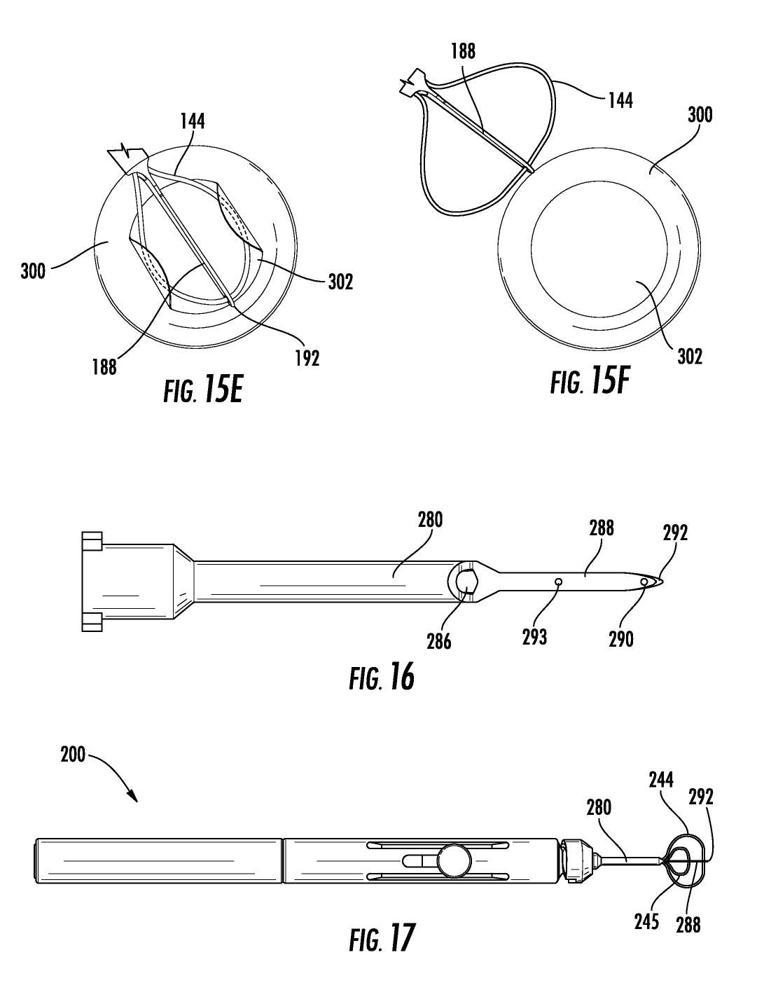

The instrument 100 may be configured to be disposed within, or between, the scroll-like rolls of the tissue, and then expanded to unroll the tissue. A practitioner may insert a distal tip 140 of the instrument 100 within the center of a cigar-like scroll, or between the two scrolled up edges. This distal tip 140 may be manipulated by a practitioner to expand a loop 144 of wire, cable or thread material 146 to unroll the layer of Descemet's membrane and endothelial cells without damaging or destroying the cells during the unfolding process as illustrated in FIGS. 15A-F. The distal tip 140 may be manipulated by means known in the art, either mechanically or electromechanically. The distal tip 140 at the distal end 120 of the instrument may be caused to expand in a horizontal plane, to safely and effectively unscroll the transplant tissue. The distal tip 140 may be expanded in symmetrical manner, which may also allow the practitioner to unscroll the transplant tissue. While a closed end loop 144 made up of a thread 146 or similar material is contemplated for the distal tip 140, such that the loop 144 may be expanded to unscroll the tissue, other distal tips configured to symmetrically unscroll the tissue are also contemplated. For example, a distal tip end may be opened ended, such that it can splay outward to unscroll the tissue.

In an embodiment, the wire, cable or thread material 146 may be made of materials that may avoid bending permanently or become knotted or kinked if folded when the instrument is actuated forward from the hand piece 130 while the distal tip 140 has been introduced into the anterior chamber of the eye. In one embodiment the wire or thread 146 may be made of nylon. In another embodiment the wire or thread may be made of polypropylene. In another embodiment the wire or thread may be made of medical grade suture or surgical grade nylon. In another embodiment the wire or thread may be made of thin twined steel. In yet another embodiment the wire or thread may be made of a single strand of steel. In other embodiments the wire or thread may be made of a variety of types of flexible plastic or flexible rubber. In another embodiment the wire or thread may be made of any flexible metal known in the art. In another embodiment the wire or thread may be made of any variety of bendable synthetic or organic materials. In one embodiment the wire or thread may be made of durable material and is meant to be reused. In another embodiment the wire or thread may be made of disposable material and is designed to be discarded after a single use.

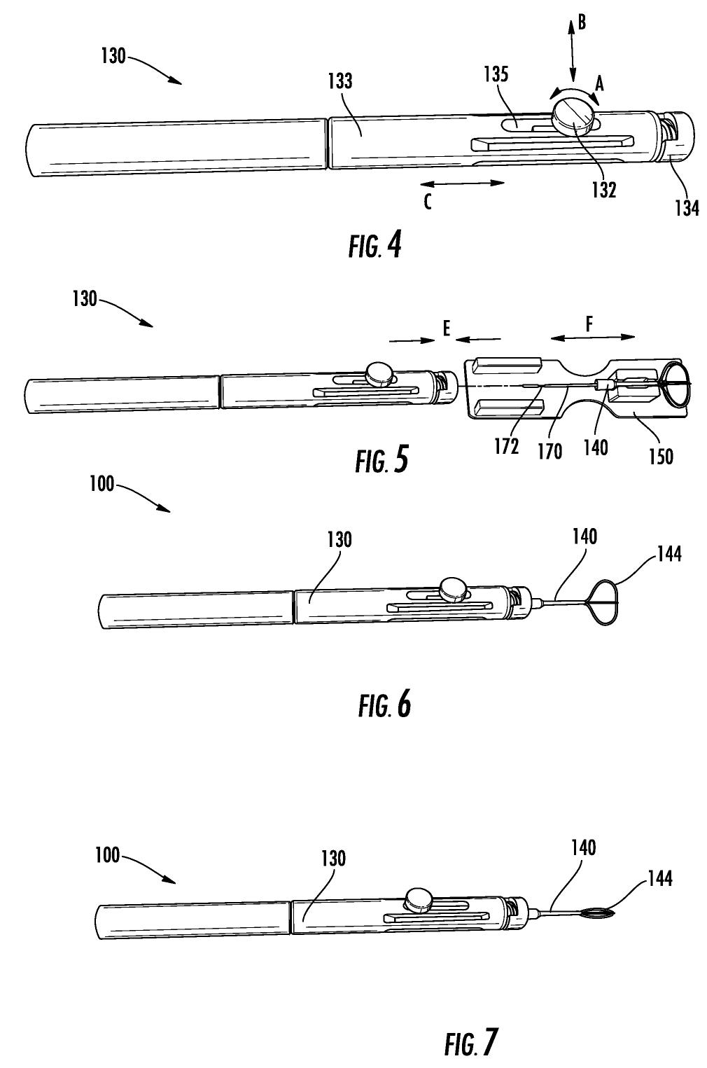

In an embodiment illustrated in FIGS. 1 and 2, an instrument 100 may have a distal end 120 which may be made of a distal tip 140 and a proximal end 110 which may be made of a hand piece or handle 130. The instrument 100 may be reusable and suitable for an autoclave or may be disposable or for single use. In an embodiment, the hand piece 130 and the distal end 140 may be unitary instrument or the distal end may be detachable. In the embodiment of the instrument 100 illustrated in FIGS. 4 through 7 the hand piece is reusable while a disposable distal tip 140 may be inserted for use prior to use in the procedure and is disposable.

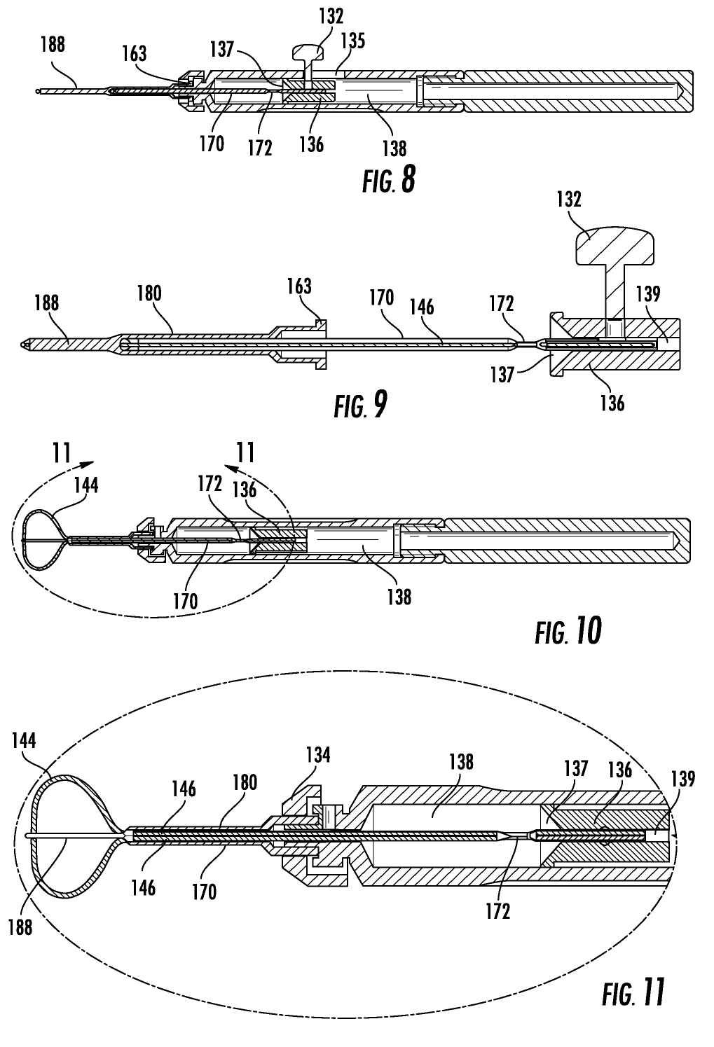

The handle 130 may have a handgrip end 131 and an actuating section 133. The actuating section 133 may include a knob, or lever, or button 132, that may be slid or translated in the lateral direction within an opening or channel 135 in the actuating section 133. Button 132 may be disengageably connected to a piston or actuator 136 disposed in a chamber 138 in the actuating section 133 of the handle 130 (shown in FIG. 8) such that manipulating the button 132 laterally within the channel 135 may cause the piston 136 to actuate laterally within the handle 130 chamber 138. Other means known in the art to actuate a piston are contemplated and the embodiment described herein is not meant to be limiting. For example, a screw or a plunger could be located at a proximal end of the handle and be capable of the same function. The button may be ergonomically positioned to make it comfortable for the surgeon to advance or retract the button while operating the instrument's distal tip 140 with it in the anterior chamber of the eye.

At a distal end of the handle 130 and actuating section 133, there may be a locking mechanism for use in connecting the distal tip 140 to the hand piece 130. Any type of suitable connecting or locking mechanism for connecting an attachment to a surgical instrument is contemplated. The distal end of the handle 130 may also have an opening 163. Opening 163 may be in coaxial communication with chamber 138 within the handle 130.

While the handle 130, as well as the cylinder 136 and chamber 138 within the handle, in an embodiment, may be cylindrically shaped, it may also be formed into a variety of shapes without departing from the spirit of the invention provided that it has a space in its center to actuated an inner tube or rod to manipulate a thread to open and close a loop. The handle 130 may be made of a variety of durable or disposable materials known in the art for surgical instruments. In one embodiment the hand piece 130 is comprised of steel. In another embodiment the hand piece 130 is made of titanium or any variety of metal alloys. In yet another embodiment the hand piece 130 is made of a suitable plastic or composite. A wide variety of materials known in the art may be used in the making of the hand piece which could be manufactured as a single piece or be comprised of several prefabricated parts to be assembled later. The hand piece may be made of any material or combination of materials (durable or disposable) which provide for the functionality of the above described instrument, without departing from the spirit of the invention. Furthermore, the present invention may be fashioned to have a variety of ports for the attachment of silicone tubes or IV tubing to allow for the addition of fluids such as balance salt solution or gas (e.g. SF6).

The distal end 120 of the instrument may include an attachable and/or disposable distal tip 140. FIG. 3 illustrates an embodiment of a distal tip 140 which may be used with the handle 130 of the instrument (with the thread 146 that may form a loop 144 not illustrated in FIG. 3 for clarity purposes). The distal tip 140 may have an inner tube 170 which may be inserted into the handle 130, a locking portion 182 that may interact with the collar 134 on the distal end of the handle 130 to hold the distal tip 140 securely to the handle 130, and a tip shaft 188. Each of these elements of the distal tip 140 may be hollow or have a chamber along a central axis such that thread 146 or other similar material could be positioned along the length of the tip shift 188, the locking portion 182 and the inner tube 170. The hollow opening through the distal tip 140 may be coaxial with the chamber 138 in the actuating section 133 of the handle 130. Moreover, inner tube 170 may be slideably inserted into tip shaft 188.

The inner tube 170 of the distal tip 140 may also have a crimp 172 in it, closing the inner chamber within it at the crimp point 172 such that any thread 146 or other similar material positioned within the inner tube 170 could be fixed to the inner tube. Other means of fixing a material found within the inside the inner tube such that any thread 146 or other similar material may include clamping, screwing, or adhesion are also contemplated and these methods are chosen without limitation to the scope of the invention.

The distal tip 140 may also have a locking portion 182 which may interact with the locking mechanism f the collar 134 of the handle. The locking portion 182 of the distal tip may include a number of protrusions 163 for use in securing the distal tip 140 to the handle 130.

The distal tip 140 may have one or more openings 186 one either side of the distal end of the tip shaft 180 which may allow a thread 146 to inserted through distal tip into the inner tube 170.

The distal tip 140 may have a needle or blade 188 extending forward from the tip shaft 180. The blade 188 may be thinner and flatter than the tip shaft 180. The blade 188 may be shaped so that a surgeon may position it within the anterior chamber of the recipient's eye and insert the blade 188 within the rolled up or scrolled up endothelial cell layer that has already been introduced into the anterior chamber. In another embodiment the instrument 100 may have a fine needle like rod or fine tube that will be introduced first, in advance to the formation of the wire loop, whose purpose will be to engage as well as stabilize the scroll of transplanted tissue.

The blade 188 may be less than approximately 10 mm in length, and in an embodiment be between approximately 7 and 5.5 mm in length and in another embodiment be between approximately 6.2 and 6 mm in length, measured to the tip of the blade 188.

The blade 188 may be approximately less than 1.2 mm in width, and in an embodiment or along a portion of its length, be between approximately 0.9 and 0.6 mm in width and in another embodiment or along another portion of its length, be between approximately 0.8 and 0.7 mm in width.

The blade may be less than 0.5 mm in depth, and in an embodiment or along a portion of it length, be between 0.4 and 0.2 mm in depth and in another embodiment or along another portion of its length, be between 0.8 and 0.7 mm in width.

The blade 188 may have a finer or more narrow extended tip 192 on its extreme distal end. This extended tip 192 may facilitate a surgeon's delicate task of positioning the blade 188 within the very thin and fragile rolled or scroll endothelial cell layer. The extended tip 192 may be approximately less than 2 mm in length. In an embodiment, the extended tip 192 may be also reduce in width and/or depth to be less than approximately 0.7 mm in width and to be less than approximately 0.3 mm in depth.

The blade 188, with a closed loop thread, may be made of a shape, size and variety of materials known in the art that may be small enough to fit within the central scroll of the transplanted endothelial tissue layer 302.

The components of the distal tip 140 may be comprised of any materials used in manufacturing of surgical instruments commonly known in the art. In one embodiment the inner tube 170 may be made of stainless steel. In a second embodiment the inner tube may be made of a plastic material. In another embodiment the inner tube may be made of aluminum. In yet another embodiment the inner tube may be of an alloy of multiple metals or materials. In one embodiment the inner tube may be made of durable material and be meant to be reused. In another embodiment the inner tube may be made of disposable material and be designed to be discarded after a single use. The inner tube 170, and the other components of the distal tip 140, could comprise any material (or combination of materials) which may provide for the functionality of the above described instrument 100 without departing from the spirit of the invention.

In another embodiment of the instrument 100 described herein, the extended tip 192 may be modified to have a triangular shaped tip which may be disposed on the outermost end of the distal end where the opened loop may form. A triangular tip may prevent the thread from opening the transplanted tissue 302 in an asymmetric fashion. In other embodiments the tip may be shaped as a wide variety of shapes and may not necessarily be triangular. It is envisioned that the extended tip may take any shape that is in line with the above described functionality without departing from the spirit of the present invention. The tip may also help to prevent the loop 144 from kinking, as kinking may occur while the thread is in a closed loop position.

The instrument may act to seal the surgical wound so that the anterior chamber of the eye does not collapse or may be maintained at a desired volume once the blade 188 of the distal tip 140 of the instrument 100 is inserted into the eye. This can be determined by the practitioner's preference by adding or removing balance solution to the eye through the side port of the surgical incision.

The blade 188 and the extended tip 192 of the instrument 100 described herein may be configured to accommodate different size incisions into the eye (e.g. 2.56-3.25 mm). This may produce a tight seal enabling control of the depth of the anterior chamber. In one embodiment the blade 188 and/or the extended tip 192 of the instrument 100 may be adjustable to accommodate a variety of incision sizes. In another embodiment of the blade 188 and the extended tip 192 of the instrument 100, the blade 188 and the extended tip 192 of the instrument 100 may be removable and replaceable with a variety of different sized tips allowing for different sized incisions. Either of these configurations can be used without departing from the spirit of the present invention.

The blade 188 may have a have a hole or opening 190 passing through the blade. The hole may pass through the wider and flatter width of the blade, in an embodiment, however, that positioning is without limitation to the scope. The hole 192 may be sized and shaped such that a thread 146 or other similar material may be passed through the hole 192 and be inserted into opening or openings 186 in the distal tip 140.

In assembling a distal tip in an embodiment of the invention, a thread 146 which may be used to form the loop 144, may be passed through hole 190 in the blade 188. Each end of the thread may also be passed through openings 186 to enter the tip shaft 180. The thread may then pass through the locking portion 182 of the distal tip 140 and also may enter the inner tube 170. Where the inner tube 170 is crimped 172, closing the passage way within it at the crimp point, the thread 146 may be fixed to the inner tube. The inner tube may thus be secured to the distal tip 140 by the thread 146. Given sufficient strength and rigidity to the thread, sliding the inner tube laterally (Arrow F of FIG. 5) may pull the thread 146 taut, closing the loop, or force the thread 146 to expand horizontally, forming an open loop. The wire or thread 1465 used may be comprised of any material (or combination of materials) and threaded in a variety of configurations which provides for the functionality of the above described instrument without departing from the spirit of the invention.

The length of the blade 188 and the positioning of the hole 192 may also determine the size and shape of a corresponding loop 144 of thread 146 or similar material in operation of the instrument 100. For example, the closer together hole 192 and opening 186 are, the wider or more ovoid in the perpendicular axis the loop 144 may be. In an embodiment, the thread 146 may translate freely through hole 192. In other embodiments, the thread 146 may be fixed to the blade 188. In one embodiment the loop may be formed by forces applied against the back surface of the eye as well as the expansion of the material once thread is delivered forward by the instrument. In another embodiment the shape of the wire loop is determined in the manufacturing process and the wire loop merely returns to this shape once extended from the instrument.

FIG. 4 illustrates a handle 130 prior to insertion of a distal tip 140. The button 132 may rotate counterclockwise and clockwise (Arrow A) which may have the effect of raising and lowers the button (Arrow B) such that it may be engaged with or disengaged from cylinder 136 within the chamber 138 of the handle 130. Other means of disengaging the button are contemplated, and a threaded button is not meant to be limiting. The button 132 may also be translated laterally (Arrow C) within or along channel 135, such that when the button is engaged with cylinder 136, the cylinder will also translate in the lateral direction within the chamber 138 of the handle 130. Collar 134 may rotate counterclockwise and clockwise in a plane perpendicular to the button to engage and disengage the locking components to secure the distal tip 140 to the handle 130.

The distal tip 140 may be delicate and the thread may be susceptible to kinking. The distal tip 140 may be designed to prevent the thread 146 from any kinking that may occur due to its positioning in a closed loop position. As such the distal tip 140, blade 188 and thread 146 wire may be made in a variety of disposable/reusable combinations of materials without departing from the spirit of the present invention.

In order to protect the distal tip in transport and shipping, protective packaging may also be provided that may maintain the thread 146 in an open loop form. This packaging may take the form of the blister pack 150 illustrated in FIGS. 14A and 14B. The blister pack may have protrusions 152 that may secure and protect the loop 144 and the blade 188 and also protrusions 154 that may secure the tip shaft 180 in place. When secured in a blister pack 150, distal tip 140 may also be positioned to be inserted into the handle 130 as illustrated in FIG. 5 (Arrow E). The blister pack 150 may provide a stable and secure base to facilitate insertion.

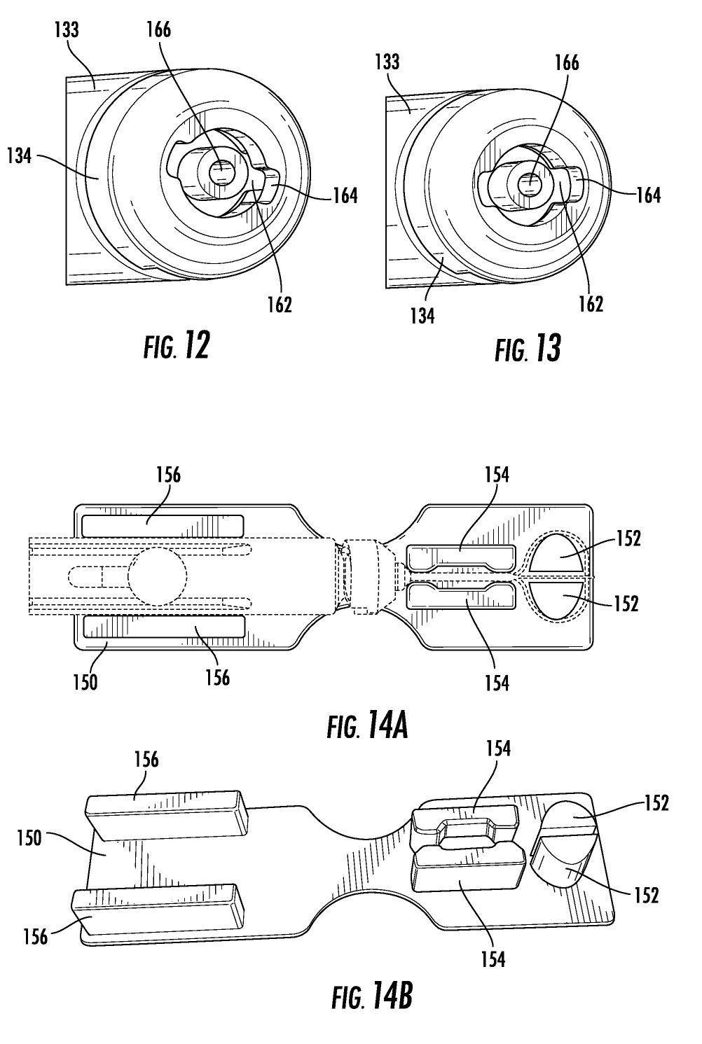

To insert the distal tip 140, a practitioner may unscrew the button 132 on the handle 130, disengaging the button. The handle 130 may be aligned with the distal tip 140 as illustrated in FIG. 5. Collar 134 may be rotated into the open position (FIG. 13). The blister pack 150 may also have protrusions 156 to secure the handle 130 during insertion, which may prevent damage to the distal tip 140 during the process. The inner tube 170 of the distal tip 140 may be inserted in opening 166 on the center of collar 134 and enter the chamber 138 within handle 130 while the handle 130 and distal tip 140 may both be supported by the blister pack 150 (FIG. 14A). As illustrated in greater detail in FIGS. 8-11, when inserted into the handle 130, the inner tube 170 of the distal tip 140, may pass through chamber 138 within handle 130 and engage hollow 139 in the cylinder 136.

As described further below, protrusions 163 in the locking portion of the distal tip 130 may pass through outer grooves 164 on collar 134 and fit within inner grooves 162 on the collar 134.

In one embodiment, the distal tip 140 may be disposable and replaceable and the thread may be threaded through the distal tip during manufacturing and may simply be replaced by the practitioner as a single thread 146 and distal tip 140 between uses. In another embodiment, the distal tip 140 may be reusable and the wire may be threaded by the practitioner prior to use. In another embodiment, the distal tip 140 may be disposable and wire may be threaded by the practitioner prior to use.

The collar 134 may then be rotated to lock the distal tip 140 in place, the locked position illustrated in FIG. 12. The button 132 may be pushed to its forward position in the channel 135 and screwed down to engage the cylinder within the handle 130. With the distal tip 140 securely attached to the handle 130, the instrument may be removed from the blister pack 150. As illustrated in FIG. 6, with the button 132 in the forward position, the loop 144 on the distal tip may be an open loop. As illustrated in FIG. 7, with the button 132 in the rear position, the loop 144 on the distal tip may be a closed open loop. This closed loop position of the instrument 100 before a practitioner may commence use for unrolling the transplant tissue.

In an embodiment, actuating the instrument to advance the thread forward to form an open loop may result in in a smooth and symmetrical expansion of the thread in a circular or semi-circular configuration that may ultimately spread the endothelial tissue as evenly as possible. The length of the blade 188 and the positioning of the hole 192 may also determine the size and shape of a corresponding loop 144 of thread 146 such that an open loop may be more or less circular, elliptical, or ovoid.

FIG. 8 illustrates a cross-sectional side view of the instrument 100 taken generally across line 8 in FIG. 2. FIG. 9 is a partial cross-sectional side view of a distal tip 140 engaged with the cylinder 136 within the inner chamber 138 of the handle, also taken generally across line 8 in FIG. 2. FIG. 10 illustrates a cross-sectional top view of instrument 100 taken generally across line 10 in FIG. 1 and FIG. 11 is an enlarged view for greater detail of the distal end of the instrument 100 as shown in FIG. 10.

In an embodiment illustrated in FIGS. 8 and 9, when attaching the distal tip 140 to the handle 130, the inner tube 170 of the distal tube may be inserted through opening 164 into chamber 138 of the handle where it may meet the cylinder 136. Cylinder 136 may have a conical or funnel shaped structure 137 at its distal end which may facilitate the inner tube 170 entering a hollow core 139 in the cylinder 136. Cylinder 136 may be a birdcage and needle cage type piston and may hold the inner tube 170 in place via tension, friction, engagement with the button 132, or by other means known in the art. Once the inner tube is engaged with the cylinder, sliding the button 132 to actuate the cylinder 136 may also actuate the inner tube 170 in the same direction.

In an embodiment, the inner tube may be connect to the hand piece 130 rather than to the distal tip. In another embodiment, a rod may be used rather than an inner tube, such that the rod may be actuated, forcing a wire or thread into an open shape form.

In an embodiment illustrated in FIGS. 10 and 11, the cylinder 136 may be in a forward position, actuating inner tube 170 of the distal tip 140 forward as well. Thread 146 may be fixed to inner tube 170 at a crimp point 172 and runs through the center of the inner tube, exiting the inner tube and the tip shaft 180 at opening 186. When inner tube 170 is pushed forward, so is thread 146, forcing the thread to expand outwardly forming the open loop 144 shown in the FIGS. 10 and 11.

In an embodiment illustrated in FIGS. 12 and 13, a circular bayonet locking mechanism may be used. In an embodiment, at the distal end of the handle 130 may be a rotatable collar or nut 134. This nut 134 may be rotatable about a central axis along the length of the handle 130. FIGS. 12 and 13 illustrate distal end of a handle of the instrument with the locking mechanism in the closed and open positions respectively. In the closed position, an inner ring groove 162 may not align with an outer ring groove 164. Rotating collar 134 counter-clockwise from the closed position to an open position may align the inner and outer ring grooves 162, 164 such that a distal tip protrusion (163 in FIG. 3) may pass through outer ring groove 164 and be set in inner ring groove 162. By rotating collar 134 clockwise, once again misaligning grooves 162 and 164, the collar 134 may hold the distal tip protrusion 163 in place. FIG. 8 illustrated the interaction between the grooves 162 and 164 in the locking collar 134 of the handle and the distal tip protrusions 163 which are engaged by the locking mechanism to hold the distal tip 140 in place.

Disclosed is an apparatus to be used for unrolling the rolled up layer of endothelial cells and their basement membrane used in the DMEK ocular surgery procedure. FIGS. 15A-F illustrate how the instrument may be used for unrolling the rolled up endothelial cells and their basement membrane 302 used in the DMEK ocular surgery procedure. The two main configurations of tissue transplanted in DMEK surgery that result from peeling off the Descemet's membrane from the donor tissue are: a double scroll and a cigar like single scroll. FIG. 15 illustrates the cigar like single roll 302, but the instrument may produce the same results and provide the same benefits for either type of roll.

The instrument 100 may be configured to have a tip disposed within, or between, the scroll-like rolls of the tissue 302, and then expanded to unroll the tissue 302. A practitioner may insert a distal end 120 of the instrument 100 within the center of a cigar-like scroll, or between the two scrolled up edges. This distal end 120 may be manipulated by a practitioner to expand to unroll the DMEK graft without damaging or destroying the endothelial cells during the transplant as illustrated in FIGS. 15A-F.

FIG. 15A illustrates the cigar roll transplant tissue 302 of Descemet's membrane with endothelial cells (DMEK graft) already in place in the anterior chamber of the eye, under the existing layer of corneal stromal tissue 300. FIG. 15B illustrates a distal tip 140 of the instrument inserted through an incision into the anterior chamber of the eye. In an embodiment, the distal tip 140 may include a blade 188 with an extended tip 192 and also be threaded with thread that is in a closed loop position. FIGS. 15 C and D illustrates the distal tip 140 acting on the transplant material 302. The shape and size of the blade 188 with an extended tip 192 may allow a surgeon to enter the center of the rolled up transplant tissue 302 and may also serve the stabilize the tissue 302 during the procedure.

Once the distal tip has been positioned within the center of the rolled tissue, the instrument may be actuated by the surgeon. While several embodiments of instruments capable of actuating a wire or thread forward into an open loop have been disclosed herein, providing a distal tip with a openable and closable loop to unroll the transplanted tissue is not intended to be limited by the means of actuation. The attachment of the thread to a distal tip of the blade may result in the thread forming an open loop (FIG. 15E). This opening of the loop may be a symmetrical and generally circular or elliptical expansion of the loop. The expansion of the loop may result in flattening of the scrolled transplanted tissue 302 in the anterior chamber of the eye. The open loop may also stabilize the DMEK graft and center the transplant tissue in the anterior chamber of the eye. This precise orientation of the DMEK graft may allow the practitioner to position the tissue 302 against the posterior surface of the cornea 300 and to complete the transplantation of the thin layer of corneal tissue.

The instrument may be removed from the anterior chamber with the loop having been returned to the closed position by the surgeon. The instrument may be capable of opening and closing repeatedly in the case that the surgeon may been to make further adjustments to unscroll and center the tissue. (FIG. 15F).

In an alternative embodiment illustrated in FIGS. 16 and 17, a distal tip 240 may be capable of opening two concentric loops. The blade 288 may have a first hole 292 to pass a thread 244 through to form an out loop and may also have a second hole 280 to pass a second thread through to form an inner loop 245. Each thread may pass through opening 286 to enter tip shaft 280 so that the distal tip may be manipulated to open and close loops 24 and 245. The inner concentric loop may help to further stabilize and position the transplanted material.

In yet another embodiment, the needle-like rod may comprise may comprise the operative portion of the instrument configured to unscroll the tissue. The needle-like portion may be configured to expand in an open-ended way (as opposed to a closed-end loop) to similarly unscroll the tissue allowing proper placement of the donor tissue. In this embodiment, a practitioner may manipulate the distal end of the instrument by use of the hand piece in a similar fashion to manipulating a closed-end loop to flatten the tissue.

In another embodiment, the hand piece or handle 130 will have provisions for irrigation of the anterior chamber of the eye. For example, a central lumen may be provide whose purpose would be to add irrigation or fluid to keep the anterior chamber of the eye formed or inflated during the surgical procedure, including during the unscrolling or flattening of the tissue.

In another embodiment the hand piece or handle 130 may have provisions for a central lumen whose purpose would be to manipulate the scroll of corneal endothelial tissue once inside the eye. In yet another embodiment of the present invention a central lumen, built into the instrument's hand piece 130 could also serve to deliver air or SF6 gas as can be done during the surgical procedure in an attempt to tamponade the corneal tissue 302 against the back surface of the corneal stroma 300, the desired final position.

In another embodiment, the hand piece 130 is made of a durable, reusable material, and is sterilized between uses by the practitioner. In yet another embodiment the instrument 100 is made of a disposable material designed for single use. The handle 130, the tip 140, the wire 146, the tube 170, the shaft 180, the chamber 139 and the piston 136, and any other component described herein (or conceived of later) that would form the instrument 100 described above can be made of any material (or combination of materials) which provides for the functionality of the present invention without departing from the spirit of the invention.

In another embodiment, there may be two rods, each rod having small levers attached thereto and each may be advanced and retracted by moving the corresponding levers forward and backward. Other means than levers known in the art to advance and retract the distal end of the instrument are also contemplated. The rods may complement each, one fitting inside the other within the housing of the hand piece 130. An inner rod with a forward lever may have a very fine wire attached to its tip. The posterior ends of the wire may be attached to the outer rod. When the forward lever is advanced, the inner rod with the wire attached to its tip, may pull the outer rod with the ends of the wire attached to its outer covering. The second lever then moves proportionally forward as a unit. Once the DMEK graft comprised of Descemet's membrane and endothelial cells is introduced into the anterior chamber of the eye, the first rod may be placed in position inside the scroll of corneal tissue, the second lever may then advanced slightly by the practitioner to push the wire forward. The fixed attachment of the wire to the distal tip of the inner rod may result in the wires forming a circle when the instrument is actuated. This symmetrical circular expansion of the wire may result in flattening of the scrolled transplanted tissue in the anterior chamber of the eye. This configuration allows the practitioner to then position the tissue against the posterior surface of the cornea and to complete the transplantation of the thin layer of corneal tissue.

Other embodiments, such as the use of a hand piece having a single rod or a single lever are contemplated and do not depart from the spirit of the invention. These embodiments may facilitate use by a practitioner or provide lower cost or simpler manufacturing of the device.

It is to be understood that the above description is intended to be illustrative, and not restrictive. For example, the above-described embodiments may be used in combination with each other. Many other embodiments will be apparent to those of skill in the art upon reviewing the above description. The scope of the invention therefore should be determined with reference to the appended claims, along with the full scope of equivalents to which such claims are entitled.

What is claimed:

1. An instrument for unrolling and flattening transplanted corneal tissue, said instrument comprising,
a handle housing at least a first actuator, said at least first actuator configured to advance in a distal direction and retract in a proximal direction by operation of a user;
a distal tip for penetrating a corneal incision into an anterior chamber of the eye;
at least one wire, said at least one wire fixed to a point on said distal tip on a distal end and fixed to said at least a first actuator on another end;
wherein said at least a first actuator is advanced, said at least one wire expands in a horizontal plane forming a opened loop; and
wherein said at least a first actuator is retracted, said at least one wire retracts closing said loop.

2. The instrument of claim 1 wherein said at least first actuator is slidingly disposed inside said handle.

3. The instrument of claim 1 wherein said at least one wire is fixed to said at least one actuator at a point within said handle and is fixed to a point on said distal tip on a distal end outside of said handle.

4. The instrument of claim 1 wherein said distal tip has an opening passing through said distal tip, said opening in a direction perpendicular to a field of motion of said at least one actuator.

5. The instrument of claim 1 wherein said at least one wire is fixed to a point on said distal tip by passing through an opening passing through said distal tip.

6. The instrument of claim 1 wherein said at least one wire is fixed to a point on said at least one actuator at two ends of the at least one wire.

7. The instrument of claim 1 wherein said at least one wire expands in a horizontal plane forming an opened loop in a direction perpendicular to a field of motion of said at least one actuator.

8. The instrument of claim 1 wherein said at least one wire is made of polypropylene.

9. The instrument of claim 1 wherein said at least one wire made of a medical grade suture.

10. The instrument of claim 1, wherein, said distal tip is further configured to being disposed in the center of transplanted corneal tissue scroll.

11. The instrument of claim 10, wherein, said corneal tissue scroll is unrolled when said at least one wire expands in a horizontal plane forming a opened loop.

12. The instrument of claim 10, wherein, said corneal tissue scroll is positioned against a posterior surface of the cornea when said at least one wire expands in a horizontal plane forming a opened loop.

13. The instrument of claim 10, wherein, said at least one wire retracts closing said loop allowing said distal tip to be removed from said anterior chamber of the eye.

14. A device for unrolling descemet's membrane with endothelial cells in a DMEK procedure, said device comprising,
an elongated handle, generally cylindrical in shape and having a longitudinal axis, a distal end having an opening, an opposite proximal end, and a controller;
said handle having a chamber extending lengthwise coaxial with the longitudinal axis of said handle, said chamber extending through to said opening at said distal end;
an actuator, said actuator slidable via said controller in said chamber extending lengthwise coaxial with the longitudinal axis of said handle;
a distal tip extending from said distal end of said handle;
a wire, wherein two ends of said wire are in slidable communication with said actuator within said chamber and a length of said wire extends through said opening of said distal end of said handle;
wherein said length of wire is fixed to a point on said distal tip, such that said length of wire expands in a horizontal plane forming a opened loop when said actuator advances distally and said open loop is closed and a portion of said length of wire retracts into said handle when said actuator moves towards said proximal end of said handle.

15. The device of claim 14, wherein, said distal tip is configured for penetrating a corneal incision into an anterior chamber of the eye.

16. The device of claim 14 wherein said length of wire is threaded through an opening in said distal tip.

17. The device of claim 14 wherein said at least one wire expands in a horizontal plane forming an opened loop in a direction perpendicular to said longitudinal axis of said handle.

18. The device of claim 14 wherein said at least one wire is made of polypropylene.

19. The device of claim 14 wherein said at least one wire made of a medical grade
